(19) United States
(12) Patent Application Publication
(10) Pub. No.: US 2024/0399139 A1
(43) Pub. Date: Dec. 5, 2024

(54) TENS-BASED APPLIANCE AND THERAPY SYSTEM FOR PAIN RELIEF

(71) Applicant: Christopher DeFlavia, Swarthmore, PA (US)

(72) Inventor: Christopher DeFlavia, Swarthmore, PA (US)

(21) Appl. No.: 18/679,852

(22) Filed: May 31, 2024

Related U.S. Application Data
(60) Provisional application No. 63/470,027, filed on May 31, 2023.

(51) Int. Cl. A61N 1/04

(52) U.S. Cl. CPC A61N 1/0456; A61N 1/0472

(57) ABSTRACT

A TENS-based therapy system includes a bridged electrode appliance for delivering a TENS therapy signal from a TENS device, without interruption in the event of a momentary loss of conductive contact between the bridged electrode appliance and body tissue. The bridged electrode appliance has a multi-path circuit having circuit portions that are in parallel, such that the skin-contacting electrodes are in parallel to another portion of the circuit that provides electrical continuity between ports of the TENS device, even when one or both of the skin-contacting electrodes loses contact with the skin. This prevents the TENS device from discontinuing delivery of the TENS therapy signal by preventing the TENS device from detecting an open circuit/circuit discontinuity in the event of a temporary loss of conductive contact with body tissue, e.g., during walking or other bodily activities, making the bridged electrode appliance suitable for use with conventional TENS devices.

### CROSS-REFERENCE TO RELATED APPLICATION

This application claims the benefit of priority under 36 U.S.C. 119 (e) of U.S. Provisional Patent Application No. 63/470,027 filed May 31, 2023, the entire disclosure of which is hereby incorporated herein by reference.

### FIELD OF THE INVENTION

The present invention relates generally to electrical stimulation systems, such as Transcutaneous Electrical Nerve Stimulation (TENS), and more particularly, to a TENS-based appliance for pain relief.

### DISCUSSION OF RELATED ART

Electrical stimulation systems, such as Transcutaneous Electrical Nerve Stimulation (TENS) devices are known in the art. Such systems generate an electrical signal (e.g., comprising electrical pulses) and deliver them to muscle groups and/or nerve areas of the body, generally to mask or otherwise decrease sensations of pain associated with that part of the body.

TENS units generally involve a battery-powered control unit having elongated wire leads using electrode pads to deliver electrical pulses to particular areas of human bodies for pain relief. More particularly, conventional electrical stimulation systems typically have a control unit wired to a set of electrodes.

Many conventional electrical stimulation systems typically have the electrodes connected directly to stimulation units, making it very difficult to treat parts of a subject's body spaced apart from the electrical stimulation unit and from each other. This can provide difficulties, for example, when the area needing treatment is a distal portion of the body, such as a foot.

The electrodes are typically integrated into adhesive pads that can be adhered to the skin, adjacent and/or spanning areas to be treated with the TENS therapy. More particularly, the electrodes are arranged in electrode pairs, and two electrodes of an electrode pair are arranged on the body, such that the control unit, electrodes of the electrode pair, and the body form a complete electrical circuitry for application of the TENS therapy.

Commonly, control units are configured such that the electrical signal associated with the TENS therapy is discontinued in the event that the continuity of the electrical circuit is broken (such that the circuit is rendered “open”), for example, if an electrode loses electrical contact with the skin of the wearer. More particularly, conventional control units sense the discontinuity of the electrical circuit as the result of a loss of contact of an electrode with the skin, and terminates the therapy cycle, and associated delivery of the electrical signal that provides the TENS therapy. Accordingly, such discontinuity commonly interrupts and ends a timer or therapy cycle associated with the TENS therapy, and requires re-starting of a timer or therapy cycle via input to the control unit to restart the therapy session after the electrical circuit's continuity has been restored, e.g., by reapplying the electrode to the skin.

What is needed is a device that is capable of providing suitable TENS therapy, even to distal portions of the body, without the need for adhesive pads and/or conductive gel attached directly to the skin, and without the discontinuation of a therapy cycle in the event of momentary discontinuity of the TENS circuit.

### SUMMARY

The present invention provides a bridged electrode appliance that is capable of providing suitable TENS therapy, even to distal portions of the body, without the need for adhesive pads, and without the discontinuation of a therapy cycle (delivery of a TENS therapy signal from a TENS unit) in the event of a momentary loss of conductive contact between the bridged electrode appliance and body tissue.

The bridged electrode appliance 100 provides a structure that forms a multi-path circuit having circuit portions that are in parallel, such that the skin-contacting electrodes (and/or any body tissue providing continuity therebetween) of the bridged electrode appliance 100 are essentially in parallel to another portion of the circuit that provides electrical continuity between the ports 202a, 202b of the TENS control unit 200, even when one or both of the skin-contacting electrodes loses contact with the skin, even when the circuit is open/discontinuous between the skin-contacting electrodes. More particularly, the bridged electrode appliance provides a parallel circuit path that includes a conductive bridge element 194 that electrically couples the conductors of wire leads 190a, 190b and ensures electrical continuity of the circuit between the ports 202a, 202b of the TENS control unit 200, even when there is a loss of electrically conductive contact between the bodily tissue (e.g., foot F) and at least one of the skin-contacting electrodes 196a, 196b. This prevents the TENS control unit 200 from discontinuing a therapy cycle/delivery of a TENS therapy signal by prevent the TENS therapy unit 200 from detecting an open circuit/circuit discontinuity in the event of a temporary loss of conductive contact with body tissue, e.g., during walking or other bodily activities.

In accordance with a certain aspect of the present invention, a TENS-based therapy system is provided that includes not only the bridged electrode appliance but also a TENS control unit including at least one electrode pair for delivering a suitable electrical TENS therapy signal (e.g., electrical pulses and/or a stimulating waveform, as known in the art) for TENS therapy purposes to a portion of the body in contact with the bridged electrode appliance when the bridged electrode appliance is operatively coupled to the TENS control unit.

In certain embodiments, the TENS-based therapy system further includes a compression sleeve (e.g., a compression sock for use with a foot) that may be worn to position and retain the bridged electrode appliance in a position adjacent (e.g., in contact with) the body (e.g., a foot). In certain embodiments, the compression sleeve may be configured to support/retain the TENS control unit, or a portion thereof.

### BRIEF DESCRIPTION OF THE FIGURES

An understanding of the following description will be facilitated by reference to the attached drawings, in which:

FIG. 1 is a perspective view of a compression sock and bridged electrode appliance in accordance with an exemplary embodiment of the present invention;

FIG. 2 is a perspective view of the compression sock and bridged electrode appliance of FIG. 1, shown assembled as a TENS-based appliance in accordance with an exemplary embodiment of the present invention;

FIG. 3 is a perspective view of the TENS-based appliance of FIG. 2, shown worn on a human foot;

FIG. 4 is a perspective view of the bridged electrode appliance and control unit of the TENS-based appliance of FIG. 2;

FIG. 5 is an exploded perspective view of multiple layers of the exemplary bridged electrode appliance of FIGS. 1, 2 and 4; and

FIG. 6 is a schematic view of a bridge circuit formed by the bridged electrode appliance's layers, and associated with the TENS-based appliance of FIG. 3.

### DETAILED DESCRIPTION

The present invention provides a bridged electrode appliance that is capable of providing suitable TENS therapy, even to distal portions of the body, without the need for adhesive pads, and without the discontinuation of a therapy cycle (delivery of a TENS therapy signal from a TENS unit) in the event of a momentary loss of conductive contact between the bridged electrode appliance and body tissue.

In accordance with a certain aspect of the present invention, a TENS-based therapy system is provided that includes not only the bridged electrode appliance but also a TENS control unit including at least one electrode pair for delivering a suitable electrical TENS therapy signal (e.g., electrical pulses and/or a stimulating waveform, as known in the art) for TENS therapy purposes to a portion of the body in contact with the bridged electrode appliance when the bridged electrode appliance is operatively coupled to the TENS control unit.

In certain embodiments, the TENS-based therapy system further includes a compression sleeve (e.g., a compression sock for use with a foot) that may be worn to position and retain the bridged electrode appliance in a position adjacent (e.g., in contact with) the body (e.g., a foot). In certain embodiments, the compression sleeve may be configured to support/retain the TENS control unit, or a portion thereof.

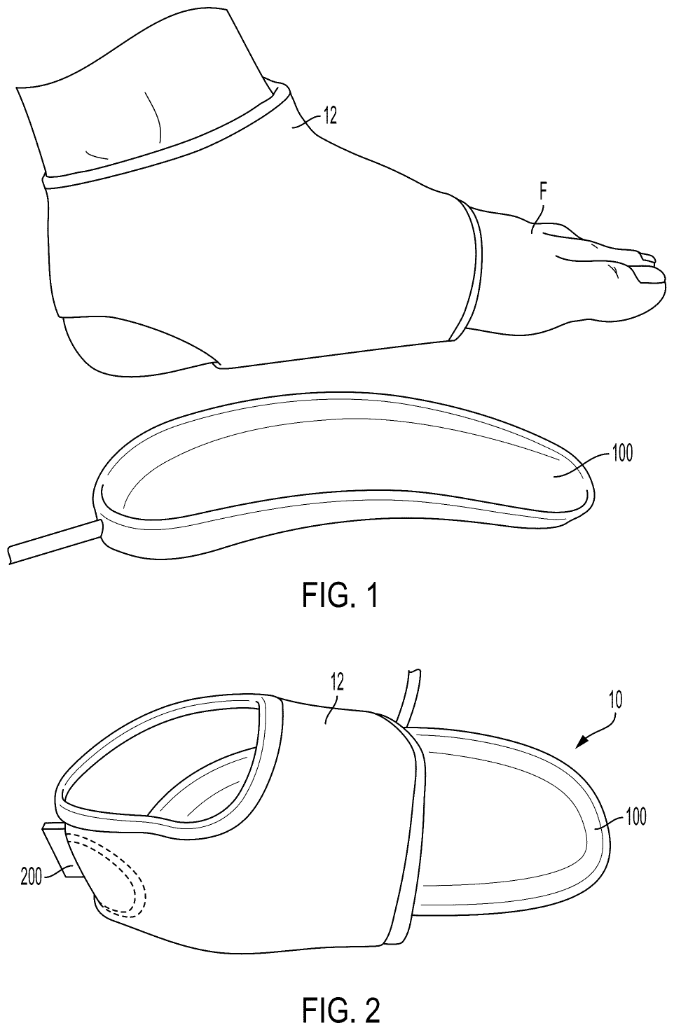

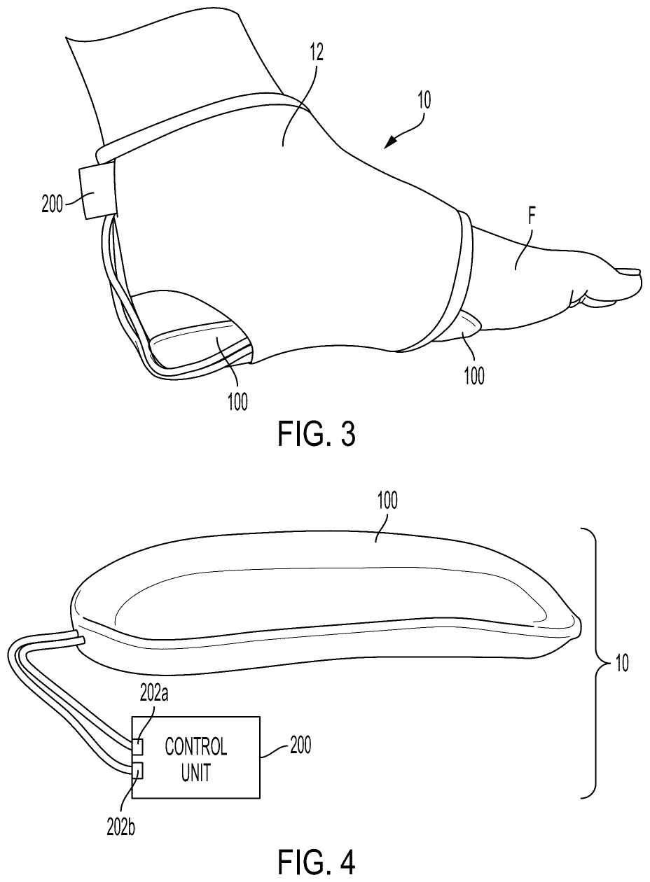

Referring now to FIGS. 1-3, an exemplary embodiment of the present invention is shown for illustrative, non-limiting purposes. In this exemplary embodiment, the TENS-based therapy system 10 is adapted for treatment of a foot, and includes a compression sleeve in the form of a compression sock 12 and an exemplary bridged electrode appliance 100, in this example, in the form of a foot sole plate.

FIG. 4 is a perspective view of the bridged electrode appliance 100 and control unit 200 of the TENS-based therapy system 10 of FIG. 2. The control unit 200 may be a conventional TENS device control unit capable of electrically coupling to a bridged electrode (sole plate) appliance 100 that includes at least one electrode pair (and suitable connectors for connecting to the TENS control using 200) for delivering a suitable electrical signal (e.g., electrical pulses and/or a stimulating waveform) for TENS therapy purposes to a portion of the body. Accordingly, as known in the art, the control unit 200 includes ports 202a, 202b for electrically coupling at least one pair of conductive wire leads that are used to deliver the suitable TENS therapy electrical signal to bodily tissue for TENS therapy purposes. Notably, the control unit 200 may be of a conventional type that is configured to interrupt and terminate a therapy session (i.e., stop delivering the TENS therapy electrical signal) in the event that the continuity of the electrical circuit is broken (such that the circuit is rendered “open”), for example, if an electrode loses electrical contact with the skin of the wearer. The control unit 200 is connectable to at least one pair of conductive wire leads of the bridged electrode appliance 100, which includes electrodes operable to abut the wearer's skin and deliver the TENS therapy electrical signal to the wearer's skin/body between the electrodes, as described in greater detail herein.

As known in the art with conventional TENS therapy and associated TENS control units, such devices generally include a pair of electrodes that are applied the wearer's skin, and then are used to deliver the TENS therapy electrical signal to the wearer's skin/body by using the wearer's skin/body to form part of the conductive circuit for the applied electrical signal. Accordingly, the wearer's body provides conductive continuity and effectively closes the electrical circuit between the electrodes. As a result, any loss of conductive contact between the wearer's body and the electrode results in an electrical discontinuity. In many TENS therapy devices, the control unit detects this disruption of electrical continuity and resultingly terminates the therapy session and delivery of the TENS electrical signal, such that subsequent restoring of electrical discontinuity does not result in continued delivery of the TENS electrical signal. Accordingly, restarting of a timer or TENS therapy session via interaction with the control unit is typically required to restart the TENS therapy, which can be undesirable, especially if the TENS control unit (and its associated power/control buttons) is not readily accessible and/or if the discontinuity occurs frequently, e.g., in association with each step while walking, etc.

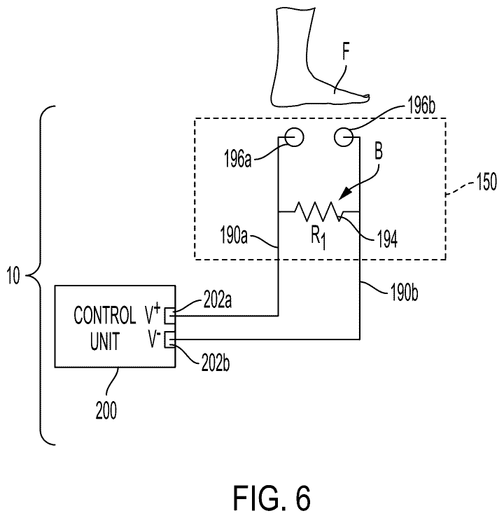

The bridged electrode appliance 100 provides a structure that forms a multi-path circuit having circuit portions that are in parallel, such that the electrodes (and/or any body tissue providing continuity therebetween) are essentially in parallel to another portion of the circuit that provides electrical continuity between the ports 202a, 202b of the TENS device/control unit 200, even when one or both of the skin-contacting electrodes loses contact with the skin, such that the circuit is open/discontinuous between the skin-contacting electrodes. FIG. 6 is a schematic view of a multi-path circuit formed by the bridged electrode appliance's layers. As will be appreciated from FIG. 6, the conductive wire leads 190a, 190b electrically coupled to the ports 202a, 202b of the TENS control unit 200 provide an electrical signal to conductive skin-contacting electrodes 196a 196b, and bodily tissue (e.g., of a foot F) may be used to complete the circuit path between electrodes 196a, 196b. Additionally, however, there is provided a parallel circuit path B that includes a conductive bridge element 194 that electrically couples the conductors of wire leads 190a, 190b and ensures electrical continuity of the circuit between the ports 202a, 202b, even when there is a loss of electrically conductive contact between the bodily tissue (e.g., foot F) and at least one of the skin-contacting electrodes 196a, 196b. In this exemplary embodiment, for illustrative purposes only, a resistive element R1 194 is shown providing continuity for the parallel circuit path B.

Accordingly, in the event of a loss of contact between an electrode 196a, 196b and the skin of the wearer, electrical continuity of the circuit is not lost, and thus the control unit 200 does not sense a loss of continuity and does not interrupt the therapy cycle, does not discontinue delivery of the TENS therapy electrical signal, and thus there is no need to restart, reset, or otherwise interact with the control unit 200.

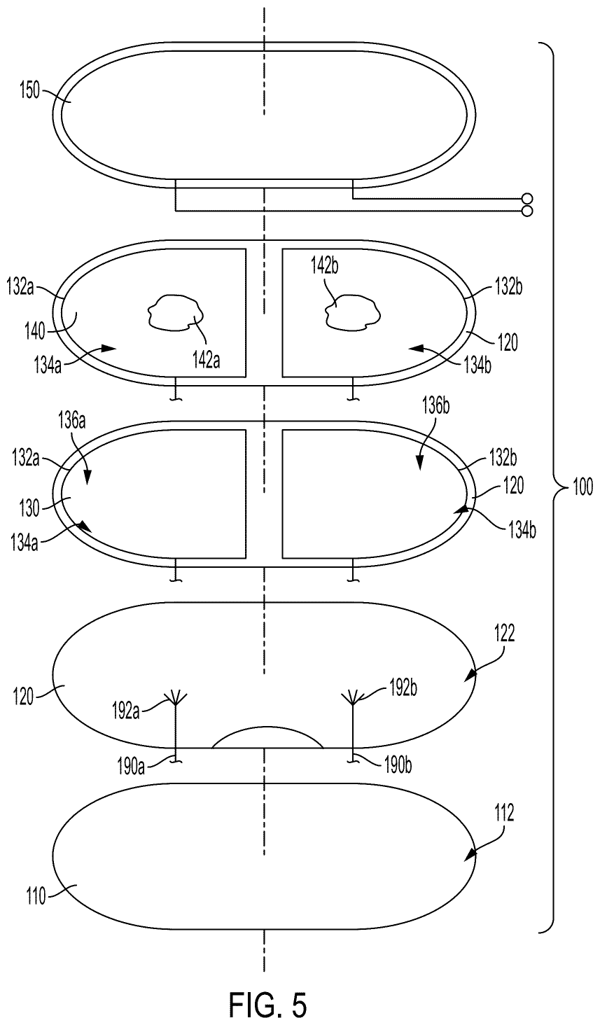

FIG. 5 is an exploded perspective view showing a successive build-up of multiple layers of the exemplary bridged electrode appliance 100 of FIGS. 1, 2 and 4. Collectively, the multiple layers 110, 120, 13, 140, 150 of the bridged electrode appliance 100 provide the multi-path circuit and electrodes of FIG. 6.

Referring now to FIG. 5, it will be appreciated the first (lower) layer of the exemplary bridged electrode appliance 100 is the base layer 110. The base layer 110 provides structural integrity for the bridged electrode appliance 100. In certain embodiments, the base layer is constructed to be relatively rigid and/or is contoured in shape to provide arch support, much like a rigid plastic orthotic insert for a foot. By way of example, the base layer 110 may be constructed using thermal beads of plastic material that may be boiled/melted and formed like a sheet, which while soft and before fully cooled/cured may be shaped to conform to the foot and/or to provide arch support to the wearer.

Referring again to FIG. 5, in this exemplary embodiment, the next layer of the exemplary bridged electrode appliance 100 is a padding layer 120, which is secured (e.g., adhered) to a top side 112 of the base layer 110. The primary function of the padding layer 120 is to provide cushioning to the foot, so that the wire leads are less likely to be felt on the bottom of the foot while walking. This layer may be non-conductive, and may act as a cushioning layer against the rigid base layer 110. In this exemplary embodiment, conductors 192a, 192b of wire leads 190a, 190b are fastened to the padding layer 120, which in this example is non-conductive, so that the conductors of wire lead 190a, 190b are not electrically coupled by the padding layer 120. Optionally, the padding layer 120 may be adhered to the base layer 110.

The base layer alone, or the base layer and padding layer in combination, provides a base of the bridged electrode appliance 100.

Referring again to FIG. 5, in this exemplary embodiment, the next layer of the exemplary bridged electrode appliance 100 is the conductive contact layer 130, which in this example is a sheet layer applied to a top side 122 of the padding layer 120, and in electrical contact with the conductors 192a, 192b of wire leads 190a 190b. In another embodiment, the conductors of the wire leads 190a, 190b are placed on top of the contact layer 130 rather than beneath the contact layer 130, and an additional conductive contact layer is applied over the conductors 192a, 19b of the wire leads 190a, 190b to mechanically secure the leads in place in electrical contact with the contact layer. By way of example, the contact layer 130 may include a thin (e.g., 0.05 mm or less) conductive foil or sheet, e.g., of copper, brass or aluminum, a conductive mesh, etc. The contact layer 130 may be adhered to the padding layer 120.

In either case, the conductive contact layer 130 is formed to have portions electrically isolated from each other, e.g., in two separate pads 132a and 132b, and each of contact pads 132a and 132b is electrically coupled to a respective one of the positive and negative conductors 192a, 192b of the pair of the conductors of the wire leads 190a, 190b. Generally speaking, the contact layer pads 132a, 132b acts as electrodes and serve to provide a surface region 136a, 136b for electrically conductive contact that is greater than that of the conductors 192a, 192b of the wire leads 190a, 190b, to help ensure robust electrical contact throughout the bridged electrode appliance 100. Accordingly, in this embodiment, each of the contact pads 132a and 132b is in a circuit path with electrodes 196a, 196b for electrically coupling to the wearer's bodily tissue.

Referring again to FIG. 5, in this exemplary embodiment, the next layer of the exemplary bridged electrode appliance 100 is a conductive gel layer 140, which is applied to the top side 134a, 134b of the each of the contact layer pads 132a, 132b of the conductive contact layer 130. In this exemplary embodiment, the conductive gel layer 140 is formed in two electrically isolated portions 142a and 142b, that are physically separate from each other, and each of gel layer portions 142a and 142b is electrically coupled to a respective one of the positive and negative conductors 192a, 192b of an electrode pair, and more particularly, to a respective one of the contact layer pads 132a and 132b. Accordingly, in this embodiment, each of the gel layer portions 142a and 142b is in a circuit path with electrodes 196a, 196b for electrically coupling to the wearer's bodily tissue.

In other embodiments, the conductive gel layer 140 may be omitted. The primary function of the conductive gel layer 140 is to electrically couple the contact layer pads 132a, 132b to a next layer, which provides a parallel circuit path for the multi-path circuit, and to help to ensure continuity between the layers during walking, etc. If continuity between the contact pads 132a, 132b (and more particularly conductors 192a, 192b of wire leads 190a, 190b and the next layer providing the parallel circuit path can be otherwise adequately ensured, then the conductive gel layer 140 may be omitted.

Referring again to FIG. 5, in this exemplary embodiment, the next layer of the exemplary bridged electrode appliance 100 is a conductive layer 150, which is applied over the conductive gel layer 140 (gel layer portions 142a, 142b) and/or contact layer 130 (contact layer pads 132a/132b), and thus. The conductive layer 150 has the primary function of electrically coupling the conductors 192a, 192b of the wire leads 190a, 190b by way of the gel layer portions 142a, 142b, and contact layer pads 132a, 132b in a secure fashion, to provide a parallel circuit path (B) that will be maintained to ensure electrical continuity of the circuit connected to the TENS control unit, even if electrical coupling/contact between the bridged electrode appliance 100 and the skin/bodily tissue is lost, to prevent a loss of continuity that would result in discontinuance of the TENS therapy signal and required resetting of a typical TENS control unit device.

The conductive layer 150 is electrically conductive but also has a relatively high degree of electrical resistance, relative to the skin/body tissue (and/or any sock, etc.), which is believed to tend to make the branch of the multi-path circuit involving the foot/body part the preferred path of least electrical resistance during contact with the foot/skin, and yet the conductive layer 150 provides sufficient conductivity/continuity to prevent a loss of continuity between the ports 202a, 202b on the TENS control unit 200 in the event of a loss of contact with the foot/skin. Accordingly, the conductive layer 150 acts as the bridge element 194 and provides the parallel circuit path B (in parallel to a circuit path through the foot), of FIG. 6.

In certain embodiments, a composite polymer material (e.g., polyolefins) consisting of carbon-impregnated polyethylene, such as that available commercially in the US as Velostat™ and/or Linqstat™, is used to form the conductive layer 150. The conductive layer 150 may be adhered to the other layers and may provide structural stability to the bridged electrode appliance 100 as a whole.

The various layers of the bridged electrode appliance 100 may be sealed around the edges with adhesive tape or may be otherwise bonded or formed together into a unitary body, for structural integrity purposes.

Conductive leads 190a, 190b from the bridged electrode appliance 100 may be positioned toward the rear/heel (when worn on the foot), and the TENS control unit 200 may be supported on the compression sleeve 12. In some embodiments, the compression sleeve 12 may be provided with an opening toward the back of the heel, to allow user to first position the compression sleeve 12 on the foot, and then slide the bridged electrode appliance 100 under the foot, approaching from the heel and passing the bridged electrode appliance 100 through the opening in the compression sleeve 12.

The exemplary arrangement thereby provides a continuous circuit via the bridge element regardless of whether electrodes of the bridged electrode appliance 100 are in contact with bodily tissue.

When in use (when connected to a TENS device delivering a TENS therapy electrical signal for the leads 190a, 190b and conductors 192a, 192b) the bridged electrode appliance 100 can be configured to provide the TENS therapy electrical stimulus to a relatively large portion of the body (e.g., about ⅔ of the sole of the foot, from the heal to past the front of arch and toward the toes of the foot), to provide for robust TENS therapy to a major portion of the foot, which is advantageous.

Additionally, when bodily tissue is in contact with the bridged electrode appliance 100 (and more particularly, with the conductive layer 150), a second continuous circuit path (via the electrodes 196a, 196b (which are effectively portions of the conductive layer 150 in this exemplary embodiment) and through the bodily tissue (e.g., of foot F) is formed, in parallel to the bridge element 194. Accordingly, the electrical signal provided by the TENS control unit may be applied to the bodily tissue (e.g., on the sole of the foot from the heel portion toward the front/toe portion). Additionally, in the event of a loss of conductive contact between the bodily tissue and an electrode 196a, 196, electrical continuity of the TENS therapy circuit is maintained via the bridge element 194 (namely, the conductive layer 150).

Notably, the appliance 100 eliminates the need for adhesive gel to be placed on the person's skin as is often the case for typical TENS pads/systems, which can be undesirable, and enables the device to be worn “dry” against the skin, as a wearable device. The electrical stimulus of this TENS-based appliance for pain relief may be used to blocks the pain receptors associated with diabetic nerve pain, neuropathy, plantar fasciitis, plantar mitosis and arch pain. Additionally, the device may be used, e.g., on the sole of the foot, to improve diabetic circulatory issues, in that the stimulus can greatly improve poor blood circulation to the extremities of the foot, and avoid or lower amputation risk associated with poor blood circulation.

In certain embodiments, the control unit 200 may be integrated into the bridged electrode appliance 100, or the compression sleeve 12 or be supported on the compression sleeve 12. Additionally, an interface may be provided to control and/or provide inputs to the control unit 200 via wireless communication with a remote device, such as a smartphone running a suitable software app.

It should be noted that other bridged circuit structures may be provided in accordance with the present invention. By way of example, the contact layer may be continuous to be electrically conductive between the conductors 192a, 192. In this case, the conductive layer 150 may be retained (to provide two bridged circuit paths), or may be eliminated (to replace the bridging effect provided by the conductive layer described above). The conductive gel layer may not be employed in certain embodiments. Alternatively, conductive gel layers may be positioned elsewhere within the bridged electrode appliance.

It should be further noted that although in the illustrative example above the appliance 100 is adapted to use with the foot, that other embodiments adapted are adapted to be worn adjacent other portions of the body to deliver a TENS therapy signal to other portions of the body, as described, consistent with the present invention. Accordingly, it may not be necessary, for example, for the base layer to be rigid, in certain embodiments, or to be adapted to conform or support a foot.

In use, the bridged electrode appliance 100 may be slipped over the foot/ankle (to be worn over the foot/angle region, within a suitable ankle compression sleeve), with the appliance 100 in contact with the sole of the foot, similar to a conventional orthotic device. Wires and/or the TENS unit may be supported on the compression sleeve. Alternatively, a separate wearable strap/band may be employed to support the TENS unit (and its control buttons) in a calf region of the leg, where the ON/OFF, intensity, etc. buttons are readily accessible for use. Alternatively, the TENS unit may be configured for wireless control (e.g., ON/OFF, intensity, etc.), e.g., via a remote control, via a Bluetooth/wireless connection to a smartphone or other device, etc., for convenience and/or to eliminate the needs for a separate strap to facilitate carrying of the TENS unit on the compression sleeve.

The bridged electrode appliance 100 may then be worn in a shoe, etc., and may be turned on/off and/or be adjusted as needed while worn in the shoe, to deliver a TENS therapy signal as needed or desired. Notably, walking and other activities involving use of the foot will not result in a loss of contact with the skin/body tissue that would case a discontinuation of delivery of the TENS therapy signal from the TENS device due to a perceived loss of continuity (for TENS units configured to operate such that the TENS therapy signal is discontinued when a loss of continuity is sensed), because electrical continuity is maintained continuously by way of the bridge circuit leg B of the bridged electrode appliance 100, even when the appliance 100/conductive layer 150 is not well-coupled electrically to bodily tissue.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A bridged electrode appliance for a TENS control unit, the bridged electrode appliance comprising:
a base;
first and second contact pads disposed on said base, said first and second contact pads being electrically conductive and electrically isolated from each other;
first and second conductive leads, each of said first and second conductive leads comprising respective first and second conductors respectively coupled one of said first and second contact pads, said leads terminating in connectors configured to form electrical connections with ports of the TENS control unit; and
a conductive layer electrically coupling the first and second conductors.

2. The bridged electrode appliance of claim 1, wherein said conductive layer has an electrical resistance greater than that of human body tissue.

3. The bridged electrode appliance of claim 1, wherein said conductive layer has an electrical resistance approximately equal to that of human body tissue.

4. The bridged electrode appliance of claim 1, wherein said conductive layer provides electrical continuity and electrical resistance suitable to prevent the TENS control unit from sensing a discontinuity and discontinuing delivery of a TENS therapy signal via ports of the TENS control unit.

5. The bridged electrode appliance of claim 1, wherein said base comprises:
a base layer, said base layer being constructed of a rigid material.

6. The bridged electrode appliance of claim 5, wherein said base further comprises:
a padding layer position over said base layer, said padding layer being constructed of a cushioning material.

7. The bridged electrode appliance of claim 6, wherein said padding layer is non-conductive.

8. The bridged electrode appliance of claim 6, wherein said first and second conductive leads are fastened to said padding layer.

9. The bridged electrode appliance of claim 6, wherein each of said first and second contact pads has a respective surface region providing electrically conductive contact greater than that of a respective conductor electrically coupled thereto.

10. The bridged electrode appliance of claim 6, further comprising first and second gel layer portions disposed on said first and second contact pads, respectively, each of said first and second gel layer portions comprising electrically conductive gel.

11. The bridged electrode appliance of claim 6, wherein said conductive layer is constructed of a composite polymer material comprising carbon-impregnated polyethylene.

12. The bridged electrode appliance of claim 1, wherein said base comprises:
a base layer, said base layer being constructed of a rigid material and contoured to provide arch support when said base is worn on a foot.

13. The bridged electrode appliance of claim 1, wherein said base is configured as a foot sole plate contoured to be wearable in a shoe.

14. A TENS-based therapy system comprising:
a bridged electrode appliance for a TENS control unit, the bridged electrode appliance comprising:
a base;
first and second contact pads disposed on said base, said first and second contact pads being electrically conductive and electrically isolated from each other;
first and second conductive leads, each of said first and second conductive leads comprising respective first and second conductors respectively coupled one of said first and second contact pads, said leads terminating in connectors configured to form electrical connections with ports of the TENS control unit; and
a conductive layer electrically coupling the first and second conductors; and
a compression sleeve adapted to receive said bridged electrode appliance and to be worn on a body to hold said bridged electrode appliance in a position in contact with bodily tissue of the body.

15. The TENS-based therapy system of claim 14, further comprising:
the TENS control unit comprising positive and negative ports, the TENS control unit being adapted to deliver an electrical TENS therapy signal via said first and conductive leads while said first and second conductive leads are electrically coupled to the TENS control unit via said positive and negative ports.

16. The TENS-based therapy system of claim 14, wherein said TENS control unit is configured to receive a control signal from a remote device via wireless transmission.

17. The TENS-based therapy system of claim 14, wherein said base is configured as a foot sole plate contoured to be wearable in a shoe.

18. The TENS-based therapy system of claim 14, wherein said base is configured as a foot sole plate contoured to provide arch support when said base is worn on a foot.

19. The TENS-based therapy system of claim 14, wherein said compression sleeve is configured to support the TENS control unit 200.

20. The TENS-based therapy system of claim 14, wherein said compression sleeve is provided with an opening dimensioned to admit passage of said bridged electrode appliance into said compression sleeve.

21. A bridged electrode appliance for a TENS control unit, the bridged electrode appliance comprising:
a base layer, said base layer being constructed of a rigid material;
a padding layer positioned over said base layer, said padding layer being constructed of a cushioning material;
first and second contact pads positioned over said padding layer, said first and second contact pads being electrically conductive and electrically isolated from each other;
first and second conductive leads, each of said first and second conductive leads comprising respective first and second conductors respectively coupled one of said first and second contact pads, said leads terminating in connectors configured to form electrical connections with ports of the TENS control unit;
first and second gel layer portions disposed on said first and second contact pads, respectively, each of said first and second gel layer portions comprising electrically conductive gel; and
a conductive layer electrically coupled to said first and second gel layer portions and electrically coupling said first and second conductors.

22. The bridged electrode appliance of claim 21, wherein said conductive layer has an electrical resistance greater than that of human body tissue.

23. The bridged electrode appliance of claim 21, wherein said base layer is contoured to provide arch support when said base is worn on a foot.
